(19) United States
(12) Patent Application Publication
(10) Pub. No.: US 2015/0126619 A1
(43) Pub. Date: May 7, 2015

(54) IONIC GEL

(71) Applicants: Kjell Morten Varum, Trondheim (NO); Kurt Ingar Draget, Trondheim (NO)

(72) Inventors: Kjell Morten Varum, Trondheim (NO); Kurt Ingar Draget, Trondheim (NO)

(73) Assignee: NORWEGIAN UNIVERSITY OF SCIENCE AND TECHNOLOGY (NTNU), Trondheim (NO)

(21) Appl. No.: 14/344,929

(22) PCT Filed: Sep. 14, 2012
(86) PCT No.: PCT/EP12/68137

§ 371 Date: Nov. 17, 2014

(30) Foreign Application Priority Data
Sep. 16, 2011 (GB) 1116050.4

(51) Int. Cl. A61K 47/36; A61K 9/06

(52) U.S. Cl. CPC A61K 47/36; A61K 9/06
USPC 514/779

(57) ABSTRACT

A process for the formation of an ionic gel comprising contacting a first polyelectrolyte reactant having a backbone comprising a plurality of β-1,4-glycosidic linkages in the ⁴C₁ conformation and an oligoelectrolyte reactant or second polyelectrolyte reactant, having a backbone containing a plurality of β-1,4-glycosidic linkages in the ⁴C₁ conformation, in aqueous solution under pH conditions such that one reactant is charged and the other uncharged; adding a donor to adjust the pH such that said uncharged reactant possesses a charge opposite to that of the charged reactant so as to form an ionic gel.

The present invention relates to a novel ionic gel, in particular a chitosan-alginate ionic gel, as well as a process for the manufacture of the ionic gel and the use thereof in a variety of applications. In particular, the inventors utilise manipulation of pH, as opposed to heat, in order to develop an ionic interaction between the charged atoms of the reactants and form a pharmaceutically acceptable gel.

Chitosans are a family of biopolymers carrying positive electrostatic charges (polycations) composed of β-1,4-glycosidic-linked building units. They are derived from the highly acetylated polymer chitin and chitosan can therefore be supplied in N-acetylated or de-N-acetylated form. The polymer is made up of units of glucosamine where polymer properties such as charge density and neutral-pH solubility can be controlled by the content of the acetyl groups and the molecular weight of the polysaccharide. Chitosans are rather special among the polysaccharides since most analogues are either electrostatically neutral or poly-anions. Chitosans are therefore regarded as promising in terms of their ability to act as carrier matrixes in Drug Delivery Systems (DDS) with high adherence to biological surfaces (e.g. as scaffolds in cell growth). Generic chitosans are produced by treating chitin, a structural polymer occurring in all animals with an outer skeleton, with alkali at elevated temperatures.

Alginates are a family of biopolymers carrying negative electrostatic charges (a poly-anion) composed of two building units, β-D-Mannuronic (M) and α-L-Guluronic acid (G). Alginates with widely varying chemical composition and sequence can be extracted from different sources. An important property of alginates is their ability to form biocompatible gels with divalent cations such as calcium, which are linked to the affinity of such ions to the (consecutive) guluronate residues in the polymer chain, meaning that alginates, with low content of G do not form gels with calcium.

Whereas chitin is an abundant structural polymer, this is not the case with chitosans, implying that there are few examples from nature as to how chitosans can be gelled and solidified. A considerable amount of scientific effort has been put into the challenge of being able to prepare a chitosan based biocompatible gelling system. The proposed gelling methods have generally involved potentially toxic chemical compounds with low bio-compatibility (e.g. heavy metal ions and glutardialdehyde). Recently, a new (non-ionic) chitosan gelling system has been reported. This system is based on using a neutral-soluble chitosan (pH above 7) that upon heating forms a chitosan gel. EP1945712 describes chitosan gels formed by thermal proton transfer from the chitosan to an inorganic phosphate base.

In WO2009/056602, crosslinked chitosan gels are described in which a random acetylated chitosan is crosslinked with a bifunctional crosslinking agent. The use of bifunctional crosslinking agents is limited however by their biocompatibility. The present inventors sought to use alginates and chitosan to form gels. These are envisaged to have valuable properties as biomaterials.

However, mixtures of chitosan and alginates will usually precipitate upon mixing due to strong electrostatic interactions. Thus, there is a need for a physical and biocompatible gelling system for both low G (high M) alginates and for chitosans in general.

It has been surprisingly found that neutral-soluble (i.e. highly acetylated chitosans) when mixed with alginate oligomers at a pH where the chitosan is mainly uncharged (such as a pH>7) and upon addition of a proton-donating substance (such as GDL) do not precipitate when the pH is lowered but form chitosan-alginate ionic gels. Additionally, it has been found that alginates when mixed with neutral-soluble chitosan oligomers at a pH where the chitosan oligomers are mainly uncharged (such as pH>7) and upon addition of a proton-donating substance (such as GDL) do not precipitate when the pH is lowered but form alginate-chitosan ionic gels.

The present invention is believed to be the first description of the formation of simple ionic gels between chitosan and alginate. The interaction between these polymers has however been previously investigated.

In J Appl Polym Sci Vol 88, 346-351, Simsek-Ege et al discuss the formation of a polyelectrolyte complex between alginate and chitosan as a function of pH. This is however a precipitate rather than a gel. Gels are explicitly avoided by Simsek-Ege.

US2006/0115511 has a similar disclosure where alginate and chitosan polymers are mixed under conditions where precipitation rather than gelling will occur.

The present inventors have realised that the distance between charged amino groups in a chitosan polymer or oligomer is the same as the distance between the charged carboxyl groups in a poly-mannuronate alginate polymer or oligomer. This is due to the presence of the β-1,4-glycosidic linkage and the ⁴C₁ ring conformation in both molecules.

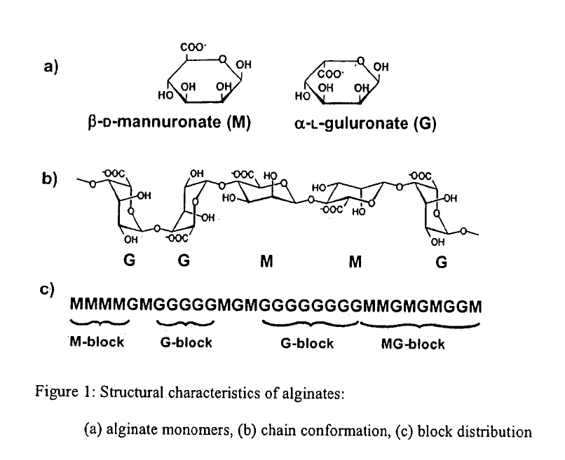

Moreover, the inventors have further appreciated that the consecutive sugars in chitosan and poly-mannuronate are rotated 180° (with the consequence that every second sugar will have a charged group pointing in the same direction) and the distance between these charged groups pointing in the same direction in any naturally occurring polyelectrolyte containing the β-1,4-glycosidic linkage in the ⁴C₁ ring conformation is the same. Thus, these polysaccharides are extended structures where the charges on every second sugar unit will point in one direction and the remaining charges will point in the opposite direction and where the charges on each side are separated by the same distance of ca 10.5 Å. This relationship is depicted in FIG. 1 b.

The principles discovered by the inventors in relation to chitosan and alginate gels in the present invention are therefore broadly applicable to any biopolymer containing the β-1,4-glycosidic linkage and the ⁴C₁ ring conformation (from hereon referred to as di-equatorial glycosidic linkages), i.e. they are broadly applicable to naturally occurring di-equatorial glycosidic linkage containing polyelectrolytes. The present inventors have realised that valuable ionic gels can be prepared using polyelectrolytes containing these di-equatorial glycosidic linkages by the manipulation of charge. In particular gels can be formed through the interaction of charged groups on the polyelectrolytes.

Unlike prior art methods, by careful control of pH, the present inventors prevent precipitation and ensure gelling. Moreover, the entire process can be carried out without the application of heat and hence without the risk of denaturing of any of the biomaterials employed. Moreover, the process avoids the use of potentially bio-incompatible crosslinking agents.

### SUMMARY OF INVENTION

Thus viewed from one aspect the invention provides a process for the formation of an ionic gel comprising contacting a first polyelectrolyte reactant having a backbone comprising a plurality of β-1,4-glycosidic linkages in the ⁴C₁ conformation and an oligoelectrolyte reactant or second polyelectrolyte reactant, having a backbone containing a plurality of β-1,4-glycosidic linkages in the 4C₁ conformation, in aqueous solution under pH conditions such that one reactant is charged and the other uncharged;

adding a donor to adjust the pH such that said uncharged reactant possesses a charge opposite to that of the charged reactant so as to form an ionic gel.

Viewed from another aspect the invention provides a process for the formation of an ionic gel comprising contacting a charged first polyelectrolyte having a backbone comprising a plurality of β-1,4-glycosidic linkages in the ⁴C₁ conformation with an uncharged oligoelectrolyte or uncharged second polyelectrolyte having a backbone containing a plurality of β-1,4-glycosidic linkages in the ⁴C₁ conformation in aqueous solution;

adding a donor to adjust the pH such that said oligoelectrolyte or second polyelectrolyte possess a charge opposite to that of the charged first polyelectrolyte so as to form an ionic gel.

Viewed from another aspect one the invention provides a process for the formation of an ionic gel comprising contacting an uncharged second polyelectrolyte having a backbone comprising a plurality of β-1,4-glycosidic linkages in the ⁴C₁ conformation with a charged oligoelectrolyte or charged first polyelectrolyte having a backbone containing a plurality of β-1,4-glycosidic linkages in the ⁴C₁ conformation in aqueous solution;

adding a donor to adjust the pH such that said uncharged second polyelectrolyte possess a charge opposite to that of the charged oligoelectrolyte or charged first polyelectrolyte so as to form an ionic gel.

Viewed from another aspect the invention provides a process for the formation of an ionic gel comprising contacting a charged first polyelectrolyte having a backbone comprising a plurality of β-1,4-glycosidic linkages in the ⁴C₁ conformation with an uncharged oligoelectrolyte or uncharged second polyelectrolyte having a backbone containing a plurality of β-1,4-glycosidic linkages in the ⁴C₁ conformation;

adding a proton donor capable of reducing the pH such that said oligoelectrolyte or second polyelectrolyte possess a charge opposite to that of the charged first polyelectrolyte so as to form an ionic gel.

Viewed from another aspect one the invention provides a process for the formation of an ionic gel comprising contacting an uncharged second polyelectrolyte having a backbone comprising a plurality of β-1,4-glycosidic linkages in the ⁴C₁ conformation with a charged oligoelectrolyte or charged first polyelectrolyte having a backbone containing a plurality of β β-1,4-glycosidic linkages in the ⁴C₁ conformation;

adding a proton donor capable of reducing the pH such that said uncharged second polyelectrolyte possess a charge opposite to that of the charged oligoelectrolyte or charged first polyelectrolyte so as to form an ionic gel.

In particular, the pH is reduced by the addition of a proton donor and said second polyelectrolyte is a chitosan polymer and said first polyelectrolyte is an alginate polymer. In particular said oligoelectrolyte is a chitosan or alginate oligomer.

Viewed from another aspect the invention provides an ionic gel obtained by a process as hereinbefore defined.

Viewed from another aspect the invention provides an ionic gel comprising a positively or negatively charged polyelectrolyte having a plurality of β-1,4-glycosidic linkages in the ⁴C₁ conformation and an oppositely charged oligoelectrolyte having a plurality of β-1,4-glycosidic linkages in the ⁴C₁ conformation.

Viewed from another aspect the invention provides an ionic gel comprising a chitosan polymer having a degree of acetylation of 40 to 70% and an alginate oligomer or polymer.

Viewed from another aspect the invention provides an ionic gel comprising an alginate polymer and a chitosan oligomer.

In all the gels of the invention, gelling is caused by charge interaction between the positive and negative charges on functional groups linked to the backbone of the electrolyte.

Viewed from another aspect the invention provides the use of an ionic gel as hereinbefore defined in drug delivery, as a bioadhesive or as a scaffold.

Viewed from another aspect the invention comprises a pharmaceutical composition comprising a gel as hereinbefore defined along with an active ingredient.

### DEFINITIONS

The invention requires the use of uncharged and charged reactants and the subsequent introduction of a charge to said uncharged reactant. It will be appreciated that the reactants have a particular pKₐ value and at a pH equal to the pKₐ-value there is per definition equal proportions of the charged and uncharged species. Depending on the nature of the charged unit, at pH-values above or below the pKₐ-value there will be increasing or decreasing proportions of the charged species. For chitosan containing an amino-group with a pKₐ-value of 6.5 therefore, at a pH of above 6.5, chitosan will carry decreasing amounts of charged units. The term charged is therefore used herein in relation to chitosan to mean that the material in question is subject to pH-conditions below its pKₐ-value. Uncharged chitosans are subject to pH conditions above the pKₐ-value, e.g. 0.5 units above the pKₐ-value. For example, at a pH-value one unit above the pKₐ-value, i.e. pH 7.5, only 10% of chitosan's amino-groups will be charged.

This definition then generally applies to other polyelectrolytes and oligoelectrolytes. For alginates, containing carboxyl groups with pKₐ-values of ca 3.5, at a pH of 3.5 there is equal proportions of the charged and uncharged species. Uncharged alginates are subject to pH conditions below the pKₐ-value, e.g. 0.5 units below the pKₐ-value. For example, at a pH-value one unit below the pKₐ value, i.e. pH 2.5, only 10% of alginate's carboxyl-groups will be charged.

In general therefore, in order to ensure a particular charge (or lack of charge), pH's at least 0.5 units above or below, preferably pHs of 1.0 unit above or below the pKₐ-value are used.

### DETAILED DESCRIPTION OF THE INVENTION

The present invention relates to new ionic gels. In order to form said gels, a polyelectrolyte is used. Polyelectrolytes are polymers whose repeating units bear an electrolyte group, i.e. a group capable of carrying charge in aqueous solution. The term polyelectrolyte is used herein to cover both negatively charged, positively charged and neutral polymers which are capable of carrying a charge at appropriate pH. The charge status of the polyelectrolyte is mentioned where important. It is preferred that any polyelectrolyte is soluble in water at the pH used at the start of the reaction (typically 7.0 or more when a proton donor is used).

The invention may also make use of an oligoelectrolyte. The term oligoelectrolyte refers to an oligomer whose repeating units contain functional groups that are either charged or capable of being charged at appropriate pH.

Oligoelectrolytes have, on average, 40 or fewer repeating units in the chain. It will be appreciated that oligomers are polydisperse and hence the number of repeating units within an oligomer represents an average of all the molecules present. It is preferred that any oligoelectrolyte is soluble in water at the pH used at the start of the reaction (typically 7.0 or more).

It must also be stressed that the products of the invention are ionic hydrogels and not precipitates. The materials formed in the present invention possess a gel strength. The gels are elastic or viscoelastic. The gels of the invention are also hydrogels and therefore incorporate water from the solution in which the gelling takes place. Water binding therefore distinguishes gels from precipitates. Precipitate formation involves a phase separation and water release. Hydrogel formation involves water binding and typically hydrogels bind water to the same extent as the polymer solution, typically more than 95%.

The polyelectrolytes of the invention are naturally occurring polymers or are derived from naturally occurring polymers, e.g. via deacetylation of chitin. All the polyelectrolytes of the invention contain a plurality of di-equatorial glycosidic linkages. By a plurality is meant at least 8 such linkages but typically a lot more than 8, such as at least 100. The number of such linkages will of course to be linked to the Mw of the polyelectrolyte.

The di-equatorial glycosidic linkage is shown in FIG. 1 in connection with a chitosan polymer. This same linkage can be found in M linkages of alginate and also in celluloses and other naturally occurring polysaccharides. The polyelectrolytes of the invention are generally based on polysaccharides therefore.

The positively charged groups on electrolytes of the invention are preferably based on amine groups, i.e. —NH₂ or —NH₃⁺ groups, and the negative groups on carboxyl groups, —COOH or COO⁻ groups. It will be appreciated that the charges which the reactants possess must be opposites of each other.

Polyeletrolytes of the invention include chargeable polysaccharides having a plurality of di-equatorial glycosidic linkages such as, chitosan, alginate, carboxymethylcellulose, polycationic celluloses and xylans (such as sulphated β-1,4-glycosidically linked xylans).

The oligoelectrolytes of the invention are preferably just oligomeric versions of the polyelectrolytes. Such oligoelectrolytes are preferred chargeable oligosaccharides such as alginate, chitosan, carboxymethylcellulose and beta-(1-4) linked xylans containing sulphate groups.

The polyelectrolytes form gels together with another polyelectrolyte of opposite charge or with an oligomer (an oligoelectrolyte) of opposite charge. This is achieved by starting with one charged reactant and one non charged reactant. By adjusting the pH to induce a charge on the non charged reactant a gel can form. Details of this process follow below.

In a preferred embodiment the invention relates to the interaction of alginates and chitosans. Alginate oligomers can be added to chitosan polymers or chitosan oligomers to alginate polymers in the process of the invention. Alginate polymers could also be combined with chitosan polymers to make gels as long as the necessary charge relationships are maintained.

The invention will be further described in relation to chitosans and alginates. The skilled man will appreciate that the preferred embodiment and general principles outlined below can be applied to any polyelectrolyte/oligoelectrolyte or indeed polyelectrolyte/polyelectrolyte blend.

### Chitosan Polymer

The chitosan polymer used in the invention should have a degree of acetylation of between 0.4 and 0.7. This means that 40 to 70% of the amino groups in the chitosan carry an acetyl group. Such deacetylated chitosans are ready prepared from chitin using well known techniques. These products can also be purchased commercially.

The degree of acetylation is important to ensure that the chitosan can be charged or uncharged, especially fully charged or fully uncharged at the preferred pH's discussed herein. Moreover, the degree of acetylation is important to ensure that the presence of charge or absence of charge is possible at pH's were the chitosan polymer is soluble in water.

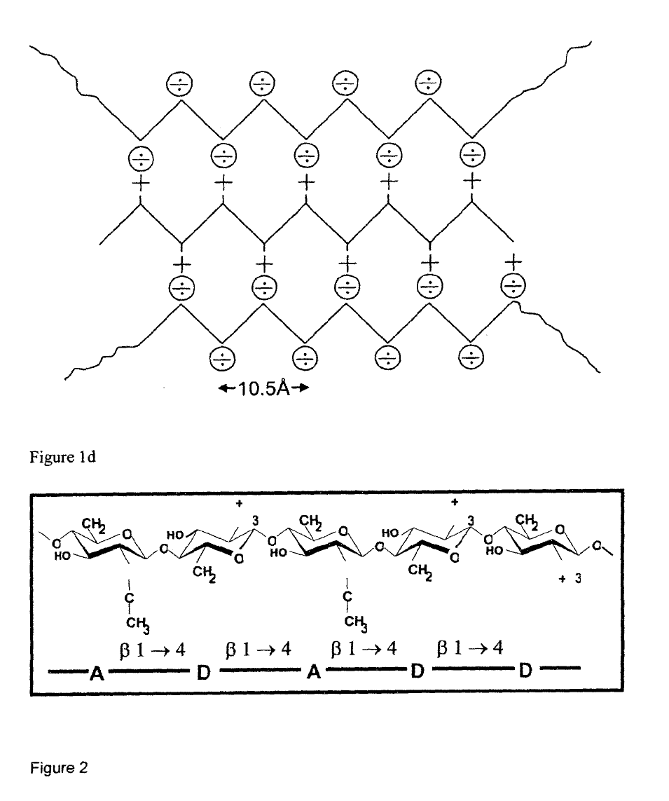

If the acetylation degree is too high the polysaccharide is insoluble in water (as chitin is). If the acetylation degree is too low, the pH at which charges are not present is incompatible with water solubility and incompatible with the oppositely charged compound with which the chitosan polymer is combined. In FIG. 2, it can be seen that at pH 6.5, with an acetylation degree of 0, chitosan polymers are not soluble. At an acetylation degree of 37%, around 20% of the chitosan is insoluble.

The degree of acetylation might also be used to control degradation of the formed gel. It is known that lysozyme degrades chitosan in the body and that the rate of degradation is directly proportional to the acetyl group content. By using therefore a chitosan with a higher amount of acetyl groups, the gel might degrade more rapidly. Alternatively, using a chitosan with fewer acetyl groups might make the chitosan more resistant to degradation. This could have important applications such as in the field of drug delivery. A material with tailorable degradation rate might be used to offer sustained release of drugs for example.

The pKₐ of the amino group of chitosan is around 6.5. It is thus preferred if the pH of the chitosan solution used in the invention is more than 6.5 preferably at least 7, preferably at least 7.5. It will be appreciated that the higher the pH, the fewer charges can be present on the chitosan. Using pH of at least 7.5 eliminates about 90% of the charges.

The chitosan polymer is preferably soluble in water at pH 6.5 or more. The term water soluble is used herein to mean a solubility of at least 10 g/L. It will be appreciated that if pH is increased too far, then the solubility of chitosan in water reduces. pH's between 6.5 and 8 are therefore preferred to ensure both no charge and water solubility.

The weight average Mw of polymeric chitosan may be in the range of 50,000 to 1,000,000.

A partially acetylated chitosan is shown in FIG. 1.

### Chitosan Oligomer

The chitosan oligomer of use in the invention preferably has, on average, 40 or fewer repeating units, such as fewer than 25 repeating units, e.g. fewer than 15 units. These repeating units are glucosamine units. It will have a minimum of 2 repeating units such as at least 3 repeating units, ideally at least 4 repeating units.

The chitosan oligomer will preferably be completely deacetylated. It will be appreciated however, that there may be low amounts of partially acetylated contamination in the oligomer used. For example up to 10 wt % of the molecules may carry an acetyl group as a contaminant. Also, the chitosan oligomer may contain a different sugar unit on the reducing end, such as can be obtained by degrading chitosan using nitrous acid resulting in a 2,5-anhydro-D-mannose unit as the reducing end unit.

The chitosan oligomer is preferably soluble in water at pH 6.5 or more and remains uncharged. It will be appreciated that if pH is increased too far, then the solubility of chitosan in water reduces. pH's between 6.5 and 8 are therefore preferred to ensure both no charge and solubility.

### Alginate Polymer

Alginates can comprise β-D-mannuronate (M) or α-L-guluronate linkages (G). In order for the gelling process of the invention to be effective, it is essential that the charged atoms are in the M form, i.e. in the di-equatorial glycosidic orientation. It is possible for the alginate of the invention to contain some G linkages as well, e.g. up to 10% of G type linkages, such as up to 5% G type linkages. This is determined using NMR as is known. Ideally, the alginate will consist of M linkages only.

The weight average Mw of the alginate may be in the range of 50,000 to 1,000,000.

The carboxyl group of alginate has a pKₐ-value of around 3.5 meaning of course that at pH's of 6.5 or more, alginate is fully charged, and the alginate polymer is soluble in water at pH 6.5 or more.

Alginates of the prevent are preferably free of multivalent metal ions.

### Alginate Oligomer

The oligomer preferably has, on average, 40 or fewer repeating unit, such as fewer that 25 repeating units, e.g. fewer than 15 units. It will have a minimum of 2 repeating units such as at least 3 repeating units, ideally at least 5 units. The oligomer is preferably free of any G units or may contain 1 such unit. The oligomer preferably has at least 6 consecutive M linkages.

The alginate oligomer is preferably soluble in water at pH 6.5 or more.

Both chitosans and alginates can be depolymerised either chemically or enzymatically to obtain the corresponding oligomers of the desired molecular size. Examples of chemical degradation mechanisms that can be used are acid hydrolysis and oxidative-reductive depolymerization. Depolymerisation of chitosan by the use of nitrous acid is a convenient way of preparing low-molecular weight chitosan, as described in for example U.S. Pat. No. 3,922,260 and U.S. Pat. No. 5,312,908. This mechanism involves deamination of a deacetylated unit, forming 2,5-anhydro-D-mannose unit at the new reducing end. Alternatively, various enzymes can also be used for depolymerization, for instance chitosanases, chitinases, and alginate lyases. Both chemical and enzymatic methods for depolymerisation will result in oligomers with a distribution of chain lengths which may, however be characterised in detail with respect to an average chain length (usually the number average) and the distribution of oligomers with respect to chain lengths. It should therefore be realized that oligomer electrolytes will usually contain a range of oligomers with varying chain lengths.

### Gelling Process

The process of the invention involves the formation of a gel from two polyelectrolytes or from a polyelectrolyte and an oligoelectrolyte. Ideally, the process of the invention involves the crosslinking of alginate polymer with a chitosan oligomer or polymer or the crosslinking of chitosan polymer with an alginate polymer or oligomer.

The techniques described below in connection with chitosans and alginates can generally be applied to all polyelectrolytes and oligoelectrolytes.

In the first preferred embodiment, a chitosan polymer is employed. This is maintained at a pH at which the chitosan polymer is uncharged. That typically means a pH of more than 6.5, such as more than 7, preferably more than 7.5.

The solvent used is typically water. Moreover, the chitosan polymer is preferably dissolved in the water. The amount of chitosan polymer added to the water may be of the order of 1 to 2 wt %. Adjustment of pH can be achieved using well known techniques. The bases and acids used should be inert to the reactants. Buffers can of course be used to establish an initial pH.

An alginate oligomer can then be added whilst the pH is maintained at a level where the chitosan is uncharged. Thus, the pH at which the alginate is added should be the same or higher than the pH of the chitosan polymer solution. The alginate oligomer can therefore be added in an aqueous solution, optionally buffered to appropriate pH.

The mixing of these two components at the pH's above does not cause the formation of a precipitate. As the alginate oligomer is charged (negatively) but the chitosan polymer uncharged there is no electrostatic interaction between the components at the pH in question.

The amount of alginate oligomer added relative to chitosan polymer can be in the weight ratio 1:2 or more, preferably 1:3 or more, such as 1:4 to 1:30. There should be an excess of the chitosan polymer relative to the alginate oligomer. In weight terms the weight ratio is preferably 1:10 to 1:25 such as about 1:20, alginate oligomer to chitosan polymer.

In order to form a gel, the pH of the material has to be lowered. This is preferably achieved slowly. The addition of a strong acid for example, reduces pH instantaneously so that instead of a gel a precipitate forms. The reduction in pH should therefore occur over a prolonged period such as minutes to hours.

This is preferably achieved by the addition of a proton donor which releases acid slowly over time. Preferably, the pH is lowered by the compound GDL as discussed below. The pH to which the blend is lowered is controlled by the amount of pH reducing additive which is added. As pH is reduced, the chitosan attracts charge (positively). The pH has to be reduced to less than 6.5, preferably less than 6, e.g. less than 5.5.

Without wishing to be limited by theory, we envisage that as the chitosan polymer attracts charge, an electrostatic interaction occurs between the di-equatorial glycosidic linkages in the alginate oligomer and in the chitosan polymer. By controlling this interaction, a gel rather than a precipitate forms.

The inventors have realised that the where a polymer backbone contains di-equatorial glycosidic linkages, the distance between the two charged functional groups, e.g. between the amino groups of chitosan or the carboxyl groups of alginate is always the same. The distance is approximately 10.5 Angstroms. There is therefore a near perfect match between the charged groups. The molecules can align as the opposite charges attract to form ionic interactions which allow the gelling process to occur.

It is especially preferred if the gelling process does not involve any heating step. The process of the invention is thus preferably carried out at room temperature. Moreover, it is preferred if the gelling process does not involve the presence of polyphosphate. Polyphosphate ions are therefore preferably absent from the process of the invention.

### Proton Donor

During the process of the invention, the pH of the blend is reduced by the addition of a proton donor. It is preferred if the proton donor realises protons slowly over time. This can be achieved, for example using glucono delta-lactone (GDL). Upon addition to water, GDL is hydrolysed to gluconic acid, and as the protons are removed from the reaction by protonation of the amino groups of chitosan (to make them positively charged), the reaction equilibrium will be forced towards the formation of gluconic acid. Thus, the amount of GDL can be used to determine the final pH of the solution. As the pH is lowered towards the pKa-value of the amino group of chitosan, this group will remove the protons.

By using this compound, the pH of the blend is reduced slowly over time and that slow release of protons prevents precipitation of the reactants and encourages gel formation.

The proton donor should not therefore be a strong acid such as HCl as those would cause rapid pH decreases and precipitation of the reactants by electrostatic interaction. The proton donor can therefore be a weak acid (i.e. one that dissociates incompletely), in particular a hydroxy acid containing a COOH group and one or more OH groups. Such hydroxy acids are preferably of low molecular weight such as less than 300 g/mol. The use of GDL slowly donating gluconic acid is particularly preferred, (i.e. HOCH₂(CHOH)₄COOH). Also of use are disguised hydroxy acids such as lactones, e.g. hydroxy lactones, e.g. having 5 or 6 atoms in a ring.

In general lactones of formula (I) may be employed:

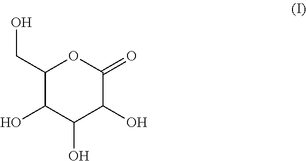 [SMILES: O=C1OC(CO)C(O)C(O)C1O]

The proton donor is conveniently added as part of a aqueous solution. The strength of that solution is not critical. A 1M solution of the donor can be used.

The amount of proton donor added will reflect the desired reduction in pH. Typically enough pH additive is added to reduce the pH to about 5 over time. It is critical to add enough donor to reduce the pH such that the uncharged species in the reaction becomes charged but not to an extent as to reduce the pH so that the alginate's carboxyl groups become uncharged. In this way, interaction between the oppositely charged species is achieved.

The interaction between the reactants is considered to be ionic as no actual bonds are formed between the reactants. Rather the gel forms due to ionic interactions between positive and negative charges. In particular, these charges are found on the di-equatorial glycosidic linkages present in the reactants.

The amount of donor added can also be used to adjust the strength of the gel by adding GDL in amounts such that the final pH is controlled between ca 6 and ca 5. The maximum charge density in between the pKa's of the gelling agents is (6.5+3.5)/2=5.

The process has been described above in relation to the reaction of chitosan polymer with an alginate oligomer. The reduction in pH introduces a positive charge on chitosan to cause gelling. The same principles apply to other similar reactions.

Chitosan polymers can also be reacted with alginate polymers using exactly the same principles as above. The relative amounts of the reactants may need to be adjusted to reflect the change from oligomer to polymer however. Thus, the amount of alginate polymer added relative to chitosan polymer may be reduced in weight terms to reflect the larger size of the alginate polymer.

The amount of alginate polymer added relative to chitosan polymer can be in the weight ratio 1:5, 1:10 or more. There should be an excess of the chitosan polymer relative to the alginate polymer.

The invention has been described in relation to the addition of an alginate oligomer to a chitosan polymer. As noted above, there is no reason why an alginate polymer could not be added to a chitosan polymer at appropriate pH, typically where the chitosan is in excess.

The invention can also be effected the opposite way round, i.e. with an excess of alginate polymer. An alginate polymer can be the most abundant material present and a chitosan oligomer can be added. The pH's values used are the same and the alginate polymer reactant carries a charge and the chitosan oligomer added does not. On reduction of pH using the proton donor, the chitosan oligomer gains charge causing gel formation. As long as one reactant is charge free and the other charged, there is no reason why a gel cannot be made by inducing a charge on the other reactant.

The amount of chitosan oligomer added relative to alginate polymer can be in the weight ratio 1:2 or more, preferably 1:3 or more. There should be an excess of the alginate polymer relative to the chitosan oligomer. In weight terms the weight ratio is preferably 1:3 to 1:10, chitosan oligomer to alginate polymer.

It is also an important feature of the invention that gels can be prepared using alginates and chitosans (or polyelectrolytes and oligoelectrolytes in general) only, i.e. without the use of an external crosslinking agent.

In general therefore, the pH before proton donor addition should be more than 6.5 but no more than 8. After donor addition, pH can be lowered to less than 6.5, e.g. to about 4, preferably to no less than 4.5, especially to around 5.

It will also be appreciated that gel formation could be achieved by increasing pH rather than reducing it. The reaction principles remain the same and assuming the necessary water solubility can be achieved starting at lower pH's, the reaction can begin from an uncharged alginate combined with a charged chitosan. On increasing the pH above the pKₐ of the alginate, the alginate attracts a negative charge and gel formation can occur.

The pH's needed for this reaction are obviously very different from those associated with pH reduction as the pH should be less than 3.5 at the start of the reaction to ensure that the alginate carries no charge. Preferably, pHs less than 3.3 are used, especially less than 3. In order to adjust the pH upwards, it will again be beneficial to avoid the use of strong bases such as hydroxides. It is preferred therefore to increase the pH slowly. The use of weak bases might therefore be appropriate such as sodium acetate.

### Gel Properties

The formed gels are preferably elastic. The formed gels are preferably viscoelastic. They may have a gel strength of at least 50 Pa, preferably at least 100 Pa. In general gels in which the polymer is chitosan are weaker than those in which the polymer is alginate, however, as noted above gel strength can be manipulated by adding more of less of the proton donor.

The gels of the invention are hydrogels as they contain water. The gels are stable at physiological pH, e.g. 5 to 8. Moreover, once the gels are formed, they are preferably not susceptible to further (moderate) pH changes and preferably resist deforming under moderate heat. Thus, even if the pH is adjusted such that one of the reactants should lose its charge, the gel preferably remains intact.

The gels of the invention will preferably contain gluconic acid as a by product of the process. This compound is pharmaceutically acceptable and there is no need to remove it.

It is a major benefit of the invention that the claimed materials are physiologically tolerable/pharmaceutically acceptable. One of the benefits of the invention is that there is no need to remove the proton donor where this is an pharmaceutically acceptable compound.

The gels can be formed into any convenient form such as beads or particles by known techniques. The gels could also be freeze dried using known procedures. Thereafter, the gels could again by formulated as beads or particles etc. The freeze dried gels are preferably rehydratable upon the addition of water.

It is believed that the degradation rate of the gels can be controlled by manipulating the amount of acetyl groups on the chitosan polymer. This may provide a sustained release option discussed below. Higher acetyl contents mean faster degradation.

The ionic gels of the invention do not interact strongly to divalent cations. it is especially preferred therefore if the gels of the invention are free of metal ions such as Ca.

### Applications

The gels of the invention have many uses. For example, they can be used in the delivery of nutraceutical or pharmaceutical compounds. Gels might be used in instant release or controlled release products. The gels could be used in drug delivery systems, e.g. in the treatment of peridontitis or in other applications where a particular pharmaceutical or a particular release profile is desired.

They can form excipients in tablets or capsules or simply be used as a gel excipient. It is preferred therefore if the gels of the invention form part of a pharmaceutical composition. Such a composition might be for oral or topical use. It can comprise at least one active ingredient, the gel and optionally any other standard excipient used in the pharmaceutical industry.

Gels could be used in their native form or freeze dried on wounds. They could be used as scaffolds for cell growth and differentiation. Of particular interest are in vitro scaffolds where the chitosan gel support is dissolved by the action of lysozyme over time. This way, a problematic relocation of cells for human tissue regeneration after in vitro growth is avoided.

The gels may be used as bio-adhesives. Gels of the invention could also be used in vaccines, tissue augmentation, cell culture, cosmetics, and as additives in the food industry and so on. The skilled man is able to perceive further applications for these novel, bioacceptable materials.

The invention will now be described with reference to the following non limiting examples and figures.

FIG. 1a shows structural properties of alginates.

FIG. 1b is a schematic illustration of ionic crosslinking in the new gelling system of the invention. In the figure a chitosan oligomer (decamer), with di-equatorial glycosidic linkages is combined with an alginate with di-equatorial glycosidic linkages. The distance between the two charged groups is always the same, 10.5 Angstroms.

FIG. 2 is a drawing of an alginate polymer showing both G and M orientation. In the M block, the distance between the charges on the COO— groups is the same as the distance between charges on the amino groups of chitosan.

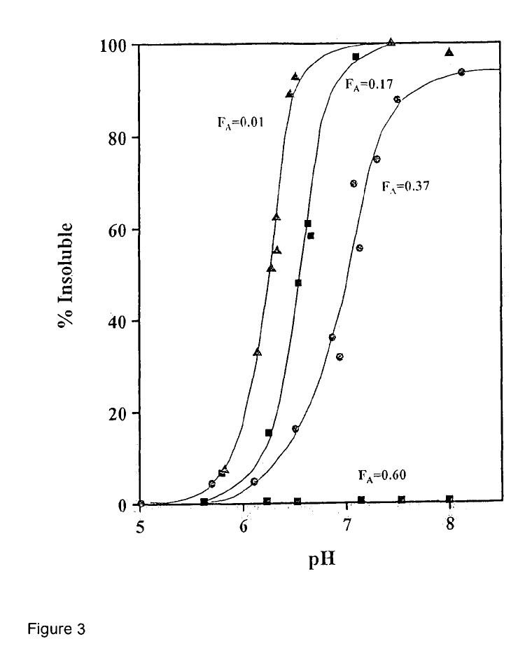

FIG. 3 shows the solubility of chitosan polymers as a function of the degree of acetylation (FA) for acetylation values less than 0.4. At these values it can be seen that achieving charged species and solubility is not possible. YES

### Gel Strength

The measurement was performed on a Reologica Stress-Tech general purpose rheometer with the following settings:
Measuring geometry: Plate/cone C 40 4 ETC fitted with a guard ring filled with low viscosity silicone oil to prevent water evaporation

### Temperature: 20° C.

### Strain: 1·10⁻³

Angular velocity: 6.28

### Breakage Test

Force at failure was determined by compression of a gel cylinder (d=15.3 mm, h=16.6 mm) using a Stable Micro Systems TA.HD.^{plus} texture analyser applying the following settings:
Probe: P/35 aluminium cylinder

### Temperature: 20° C.

Test speed: 0.1 mm/s
Trigger force: 2.0 g

### Example 1

### Chitosan Gelled with Alginate Oligomers

52.5 mg of chitosan (FA=0.44, [n]=1000 ml/g) was added to 3.5 mL distilled water and shaken overnight. 0.8 mL of 0.1M NaOH was added to the chitosan solution in steps of 0.1 mL under vigorous stirring to achieve a final pH of between 7.0 and 7.1.

1.5 g of this chitosan solution was combined with 0.5 mL of oligomers of poly-M alginate (FM=1.0, Number-average degree of polymerization (DPn) of 12). The solution of oligomers contains 2.8 mg poly-M alginate oligomers in 0.5 mL of 13 mM NaOH. This was mixed vigorously and 9.15 mg of GDL was added.

The gelling was followed by measuring the kinetics of the gelling in a rheometer, and the final G′ was 127 Pa. The pH of the gel was measured to 5.1.

### Example 2

### Alginate Gelled with Chitosan Hexamer

90 mg of alginate (Poly-Mannuronic acid with intr. visc. ([η]) of 800 ml/g) was added to add 4.5 ml distilled water and dissolved by gentle shaking (minimum 2 hours).

2.0 g of this solution was added to 27 mg of the chitosan oligomer with DP 6 and 4.5 ml distilled water added. The pH was then adjusted to 8 with NaOH.

12 mg of GDL was added to the alginate-chitosan solution with vigorous mixing. After 24 hours at room temperature, the resulting gel was measured on a Texture Analyser. Breakage at 516 g. The pH of the gel was measured to 5.

1. A process for the formation of an ionic gel comprising:
contacting a first polyelectrolyte reactant having a backbone comprising a plurality of β-1,4-glycosidic linkages in the ⁴C₁ conformation and an oligoelectrolyte reactant or second polyelectrolyte reactant, having a backbone containing a plurality of β-1,4-glycosidic linkages in the ⁴C₁ conformation, in aqueous solution under pH conditions such that one reactant is charged and the other uncharged; and
adding a donor to adjust the pH such that said uncharged reactant possesses a charge opposite to that of the charged reactant so as to form an ionic gel.

2. A process for the formation of an ionic gel of claim 1, wherein the first polyelectrolyte reactant is
a charged first polyelectrolyte and the oligoelectrolyte reactant is an uncharged oligoelectrolyte and the second polyelectrolyte reactant is an uncharged second polyelectrolyte.

3. A process for the formation of an ionic gel comprising:
contacting an uncharged second polyelectrolyte having a backbone comprising a plurality of β-1,4-glycosidic linkages in the ⁴C₁ conformation with a charged oligoelectrolyte or charged first polyelectrolyte having a backbone containing a plurality of β-1,4-glycosidic linkages in the ⁴C₁ conformation in aqueous solution; and
adding a donor to adjust the pH such that said uncharged second polyelectrolyte possess a charge opposite to that of the charged oligoelectrolyte or charged first polyelectrolyte so as to form an ionic gel.

4. A process for the formation of an ionic gel comprising:
contacting a charged first polyelectrolyte having a backbone comprising a plurality of β-1,4-glycosidic linkages with an uncharged oligoelectrolyte or uncharged second polyelectrolyte having a backbone containing a plurality of β-1,4-glycosidic linkages; and
adding a proton donor capable of reducing the pH such that said oligoelectrolyte or second polyelectrolyte possess a charge opposite to that of the charged first polyelectrolyte so as to form an ionic gel.

5. A process for the formation of an ionic gel comprising:
contacting an uncharged second polyelectrolyte having a backbone comprising a plurality of β-1,4-glycosidic linkages with a charged oligoelectrolyte or charged first polyelectrolyte having a backbone containing a plurality of β-1,4-glycosidic linkages; and
adding a proton donor capable of reducing the pH such that said uncharged second polyelectrolyte possess a charge opposite to that of the charged oligoelectrolyte or charged first polyelectrolyte so as to form an ionic gel.

6. A process as claimed in claim 1, further comprising the reaction of a positively or negatively charged polyelectrolyte having a plurality of β-1,4-glycosidic linkages in the ⁴C₁ conformation and an oppositely charged oligoelectrolyte having a plurality of β-1,4-glycosidic linkages in the ⁴C₁ conformation.

7. A process as claimed in claim 1, further comprising the reaction of a positively charged chitosan polymer or oligomer with a negatively charged alginate polymer or oligomer.

8. A process as claimed in claim 1, wherein prior to donor addition, the pH of the solution is at least 7.0.

9. A process as claimed in claim 1, wherein said donor is a hydroxy acid such as GDL.

10. A process as claimed in claim 1, where the final pH after donor addition is about 5.

11. A process as claimed in claim 1, wherein the gel forms between an alginate polymer and chitosan oligomer or a chitosan polymer and alginate oligomer.

12. A process as claimed in claim 1, wherein a chitosan polymer having a degree of acetylation of 40 to 70% is used as a polyelectrolyte.

13. A process as claimed in claim 1, wherein an alginate oligomer is used in which all units are in the M configuration.

14. An ionic gel obtained by a process as claimed in claim 1.

15. An ionic gel comprising a chitosan polymer having a degree of acetylation of 40 to 70% and an alginate oligomer or polymer.

16. An ionic gel comprising an alginate polymer and a chitosan oligomer.

17. (canceled)

18. A pharmaceutical composition comprising a gel as claimed in claim 14 along with an active ingredient.
